(19) United States
(12) Patent Application Publication
(10) Pub. No.: US 2018/0360899 A1
(43) Pub. Date: Dec. 20, 2018

(54) AGENT FOR MAINTAINING HEALTHY OBESITY

(71) Applicant: MG PHARMA INC., Osaka (JP)

(72) Inventors: Shigetoshi OKUMURA, Osaka (JP); Yuka SASAKAWA, Osaka (JP)

(73) Assignee: MG PHARMA INC., Osaka (JP)

(21) Appl. No.: 15/780,485

(22) PCT Filed: Dec. 2, 2016
(86) PCT No.: PCT/JP2016/085939

§ 371 Date: May 31, 2018

(30) Foreign Application Priority Data
Dec. 3, 2015 (JP) 2015-236693

(51) Int. Cl. A61K 36/484; A23L 33/105; A23L 33/00; A61K 36/232; A61P 3/04

(52) U.S. Cl. CPC A61K 36/484; A23L 33/105; A23V 2002/00; A61K 36/232; A61P 3/04; A23L 33/30

(57) ABSTRACT

An object of the present invention is to provide a novel agent for maintaining healthy obesity. The present invention relates to agent for maintaining healthy obesity containing processed Glycyrrhizae radix and/or Angelica keiskei as an active ingredient.

### TECHNICAL FIELD

The present invention relates to an agent for maintaining healthy obesity containing processed Glycyrrhizae radix and/or processed Angelica keiskei as an active ingredient. In addition, the present invention relates to an agent for regulating intestinal flora constituent ratio, an anti-chronic mild inflammatory agent, and an agent for reducing body weight, visceral fat, subcutaneous fat or ectopic fat containing processed Glycyrrhizae radix and/or processed Angelica keiskei as an active ingredient.

### BACKGROUND ART

Examples of diseases intimately related to obesity include heart disease, diabetes and stroke caused by hypertension and abnormally high blood sugar and cholesterol levels. However, there are known to be many people who are considered to be obese that do not exhibit these abnormalities, and are referred to as healthy obese (Non-Patent Document 1).

In recent years, attention has been focused on the intimate relationship between the constituent ratio between bacteria belonging to the phylum Bacteroidetes and bacteria belonging to the phylum Firmicutes present in human intestinal flora and human health (physical constitution and physical condition).

During obesity in humans, although the constituent ratio of bacteria belonging to the phylum Bacteroidetes decreases, as body weight decreases, the constituent ratio of bacteria belonging to the phylum Bacteroidetes increases while the constituent ratio of bacteria belonging to the phylum Firmicutes is conversely known to decrease. Slender persons are known to have a higher constituent ratio of bacteria belonging to the phylum Bacteroidetes and lower constituent ratio of bacteria belonging to the phylum Firmicutes in comparison with overweight people (Non-Patent Document 2).

Moreover, when the intestinal flora of obese mice and the intestinal flora of mice having normal body weights were respectively transplanted into two groups of mice free of intestinal flora, body weights of mice in the group transplanted with the intestinal flora of obese mice were determined to significantly exceed the body weights of mice in the group transplanted with the intestinal flora of mice having normal body weights (Non-Patent Document 3).

In addition, research has also been reported on the relationships between the constituent ratios of intestinal flora and allergies, smoking and autism (Non-Patent Documents 4 to 6).

An agent for regulating intestinal flora constituent ratio and food or a pharmaceutical containing the same have been reported that are characterized by containing at least one type of sugar absorption inhibitor that has the effect of increasing bacteria belonging to the phylum Bacteroidetes and decreasing bacteria belonging to the phylum Firmicutes (Patent Document 1).

Obesity has recently become a problem in persons ranging from the young to the middle-aged and elderly caused by such factors as improper eating habits, lack of exercise and stress. The Japan Society for the Study of Obesity defines obesity as a pathological state in which there is excessive accumulation of fat due to such as factors as hypernutrition or lack of exercise, disease caused by obesity is present, and weight loss is required. More specifically, according to the “Guidelines for the Management of Obesity Disease 2011”, obesity is diagnosed in cases of obesity (BMI of 25 or higher) or visceral fat obesity presenting with a visceral fat area of 100 cm² or more in which health problems extending over 11 diseases either caused by or associated with obesity are present. Obesity is positioned as a precursor to diabetes and arteriosclerosis in particular, and is intimately involved with so-called lifestyle diseases.

Obesity is also considered to be a type of chronic mild inflammatory state in which macrophages have been concentrated in adipocytes that have become hypertrophied by excess energy, and inflammatory cytokines such as TNF-α or IL-6 produced by macrophages induce a chronic mild inflammatory state medicated by activation of stress signals (Non-Patent Documents 7 to 10). Crown-like structures (CLS) are histological structures that phagocytize and process adipocytes that have undergone cell death during the course of obesity by being taken up by pro-inflammatory M1 macrophages, and are known to serve as sites of interaction between parenchymal cells and interstitial cells constituting the essence of chronic mild inflammation, as well as the source of chronic mild inflammation of adipose tissue (Non-Patent Documents 11 and 12). In addition, black currant extract and resveratrol have been reported to inhibit chronic mild inflammation in diet-induced obese mice (Non-Patent Documents 13 and 14).

Research has also been reported which indicates that health can be maintained despite the presence of a certain degree of obesity by severing the link between obesity and lifestyle disease (Non-Patent Document 15).

### PRIOR ART DOCUMENTS

### Patent Documents

Patent Document 1: Japanese Unexamined Patent Publication No. 2015-127340

### Non-Patent Documents

Non-Patent Document 1: Bohm, A., et al., PLoS One, 9(7), e100391 (2014)
Non-Patent Document 2: Ley, R. E., et al., Nature, 444, 1022-3 (2006)
Non-Patent Document 3: Turnbaugh, P. J., et al., Nature, 444, 1027-31 (2006)
Non-Patent Document 4: Zongxin Ling, et al., Appl. Environ. Microbiol., 80(8), 2546-2554 (2014)
Non-Patent Document 5: Biedermann, L., et al., Inflamm. Bowel Dis., 20(9), 1496-501 (2014)
Non-Patent Document 6: Williams, B. L., et al., PLoS One, 6(9), e24585 (2011)
Non-Patent Document 7: Nicklas, B. J., et al., CMAJ, 172(9), 1199-209 (2005)
Non-Patent Document 8: Ikeoka, D., et al., Rev. Assoc. Med. Bras., 56(1), 116-21 (2010)
Non-Patent Document 9: Calder, P. C., et al., Br. J. Nutr., 106, Suppl. 3, S5-78 (2011)
Non-Patent Document 10: Ghigliotti, G., et al., Inflammation, 37(4), 1337-53 (2014)
Non-Patent Document 11: Cancello, R., et al., BJOG, 113(10), 1141-7 (2006)
Non-Patent Document 12: Suganami, T., et al., Endocr. J., 59(10), 849-57 (2012)
Non-Patent Document 13: Benn, T., et al., Nutr. Biochem., 25(10), 1019-25 (2014)
Non-Patent Document 14: Lv, Z. M., et al., Reprod. Dev., 82(4), 321-8 (2015)
Non-Patent Document 15: Sekimoto, R., et al., Proc. Natl. Acad. Sci. USA, 112(16), E2058-66 (2015)

### DISCLOSURE OF THE INVENTION

### Problems to be Solved by the Invention

An object of the present invention is to provide a novel agent for maintaining healthy obesity, a novel agent for regulating intestinal flora constituent ratio, a novel an anti-chronic mild inflammatory agent, and a novel agent for reducing body weight, visceral fat, subcutaneous fat or ectopic fat.

### Means for Solving the Problems

As a result of conducting extensive research, the inventors of the present invention found that processed Glycyrrhizae radix and/or processed Angelica keiskei increases bacteria belonging to the phylum Bacteroidetes and decreases bacteria belonging to the phylum Firmicutes, inhibits the formation of crown-like structures, and reduces body weight and visceral fat, or in other words, the inventors of the present invention found that processed Glycyrrhizae radix and/or processed Angelica keiskei are capable of maintaining healthy obesity, thereby leading to completion of the present invention.

The present invention relates to that indicated below.

(1) An agent for maintaining healthy obesity containing processed Glycyrrhizae radix and processed Angelica keiskei as active ingredients.

(2) An agent for regulating intestinal flora constituent ratio containing processed Glycyrrhizae radix and processed Angelica keiskei as active ingredients.

(3) An anti-chronic mild inflammatory agent for adipose tissue containing processed Glycyrrhizae radix and processed Angelica keiskei as active ingredients.

(4) An agent for reducing body weight, visceral fat, subcutaneous fat or ectopic fat containing processed Glycyrrhizae radix and processed Angelica keiskei as active ingredients.

(5) The agent described in any one of (1) to (4) above, wherein the processed Glycyrrhizae radix is Glycyrrhizae radix powder or Glycyrrhizae radix extract obtained by extracting Glycyrrhizae radix with aqueous ethanol.

(6) The agent described in (5) above, wherein the ethanol concentration of the aqueous ethanol is 0.1% (v/v) to 99.9% (v/v).

(7) The agent described in (5) or (6) above, wherein the ethanol concentration of the aqueous ethanol is 30% (v/v) to 70% (v/v).

(8) The agent described in any one of (1) to (7) above, wherein the processed Glycyrrhizae radix contains 0.5% by weight to 20.0% by weight of glycyrrhizic acid.

(9) The agent described in any one of (1) to (7) above, wherein the processed Glycyrrhizae radix contains 0.05% by weight to 5.00% by weight of licorice saponin H2.

(10) The agent described in any one of (1) to (7) above, wherein the processed Glycyrrhizae radix contains 0.1% by weight to 10% by weight of liquiritin.

(11) The agent described in any one of (1) to (10) above, wherein the processed Angelica keiskei contains 7.0% by weight to 10.0% by weight of Angelica keiskei chalcone.

(12) The agent for maintaining healthy obesity described in (1) above, wherein maintenance of healthy obesity constitutes maintenance of a high-normal BMI value.

(13) A food or beverage, food with function claims, food for specific health use, functional nutritional food, cosmetic, quasi-drug or pharmaceutical containing the agent described in any one of (1) to (12) above as an active ingredient.

(14) An agent for maintaining healthy obesity containing processed Glycyrrhizae radix as an active ingredient.

(15) An agent for regulating intestinal flora constituent ratio containing processed Glycyrrhizae radix as an active ingredient.

(16) An anti-chronic mild inflammatory agent containing processed Glycyrrhizae radix as an active ingredient.

(17) An agent for reducing body weight, visceral fat, subcutaneous fat or ectopic fat containing processed Glycyrrhizae radix as an active ingredient.

(18) The agent described in any one of (14) to (17) above, wherein the processed Glycyrrhizae radix is Glycyrrhizae radix powder or extract obtained by extracting Glycyrrhizae radix with aqueous ethanol.

(19) The agent described in (18) above, wherein the ethanol concentration of the aqueous ethanol is 0.1% (v/v) to 99.9% (v/v).

(20) The agent described in (18) or (19) above, wherein the ethanol concentration of the aqueous ethanol is 30% (v/v) to 70% (v/v).

(21) The agent described in any one of (14) to (20) above, wherein the processed Glycyrrhizae radix contains 0.5% by weight to 20.0% by weight of glycyrrhizic acid.

(22) The agent described in any one of (14) to (20) above, wherein the processed Glycyrrhizae radix contains 0.05% by weight to 5.00% by weight of licorice saponin H2.

(23) The agent described in any one of (14) to (20) above, wherein the processed Glycyrrhizae radix contains 0.1% by weight to 10% by weight of liquiritin.

(24) An agent for maintaining healthy obesity containing processed Angelica keiskei as an active ingredient.

(25) An agent for regulating intestinal flora constituent ratio containing processed Angelica keiskei as an active ingredient.

(26) An anti-chronic mild inflammatory agent containing processed Angelica keiskei as an active ingredient.

(27) An agent for reducing body weight, visceral fat, subcutaneous fat or ectopic fat containing processed Angelica keiskei as an active ingredient.

(28) The agent described in any one of (24) to (27) above, wherein the processed Angelica keiskei contains 7.0% by weight to 10.0% by weight of Angelica keiskei chalcone.

(29) The agent for maintaining healthy obesity described in (14) or (24) above, wherein the maintenance of healthy obesity constitutes maintenance of a high-normal BMI value.

(30) The anti-chronic mild inflammatory agent described in (16) or (26) above for adipose tissue.

(31) A composition for maintaining healthy obesity containing the agent for regulating intestinal flora constituent ratio described in (2), (15) or (25) above.

(32) A composition for reducing body weight, visceral fat, subcutaneous fat or ectopic fat containing the agent for regulating intestinal flora constituent ratio described in (2), (15) or (25) above.

(33) A composition for maintaining healthy obesity containing the anti-chronic mild inflammatory agent described in (3), (16) or (26) above.

(34) A composition for reducing body weight, visceral fat, subcutaneous fat or ectopic fat containing the anti-chronic mild inflammatory agent described in any one of (3), (16) or (26) above.

(35) A food or beverage, food with function claims, food for specific health use, functional nutritional food, cosmetic, quasi-drug or pharmaceutical containing the agent described in any one of (14) to (29) as an active ingredient.

(36) Processed Glycyrrhizae radix and/or processed Angelica keiskei for use in maintaining healthy obesity.

(37) Processed Glycyrrhizae radix and/or processed Angelica keiskei for use in regulating the constituent ratio of intestinal flora.

(38) Processed Glycyrrhizae radix and/or processed Angelica keiskei for use against chronic mild inflammation of adipose tissue.

(39) Processed Glycyrrhizae radix and/or processed Angelica keiskei for use in reducing body weight, visceral fat, subcutaneous fat or ectopic fat.

(40) A use of processed Glycyrrhizae radix and/or processed Angelica keiskei in the production of a food or beverage, food with function claims, food for specific health use, functional nutritional food, cosmetic, quasi-drug or pharmaceutical for maintaining healthy obesity.

(41) A use of processed Glycyrrhizae radix and/or processed Angelica keiskei in the production of a food or beverage, food with function claims, food for specific health use, functional nutritional food, cosmetic, quasi-drug or pharmaceutical for regulating the constituent ratio of intestinal flora.

(42) A use of processed Glycyrrhizae radix and/or processed Angelica keiskei in the production of a food or beverage, food with function claims, food for specific health use, functional nutritional food, cosmetic, quasi-drug or pharmaceutical against chronic mild inflammation.

(43) A use of processed Glycyrrhizae radix and/or processed Angelica keiskei in the production of a food or beverage, food with function claims, food for specific health use, functional nutritional food, cosmetic, quasi-drug or pharmaceutical for reducing body weight, visceral fat, subcutaneous fat or ectopic fat.

(44) A method for maintaining healthy obesity, comprising administering an effective amount of processed Glycyrrhizae radix and/or processed Angelica keiskei.

(45) A method for regulating the constituent ratio of intestinal flora, comprising administering an effective amount of processed Glycyrrhizae radix and/or processed Angelica keiskei.

(46) A method for reducing chronic mild inflammation of adipose tissue, comprising administering an effective amount of processed Glycyrrhizae radix and/or processed Angelica keiskei.

(47) A method for reducing body weight, visceral fat, subcutaneous fat or ectopic fat, comprising administering an effective amount of processed Glycyrrhizae radix and/or processed Angelica keiskei.

The present invention also relates to that indicated below.

(48) An agent for regulating intestinal flora constituent ratio containing processed Glycyrrhizae radix and/or processed Angelica keiskei as an active ingredient.

(49) The agent for regulating intestinal flora constituent ratio described in (48) above, wherein the processed Glycyrrhizae radix and/or processed Angelica keiskei is any one of those selected from the group consisting of a solvent extract of the root, stem and sap of Glycyrrhizae radix and/or Angelica keiskei, the sap of Glycyrrhizae radix and/or Angelica keiskei, and a concentrate of the sap of Glycyrrhizae radix and/or Angelica keiskei.

(50) The agent for regulating intestinal flora constituent ratio described in (48) or (49) above for incorporating in a food or beverage, food with function claims, food for specific health use, functional nutritional food, cosmetic, quasi-drug or pharmaceutical.

(51) The agent for regulating intestinal flora constituent ratio described in any one of (48) to (50) above for restoring and/or stabilizing healthy intestinal flora.

(52) A composition for improving intestinal flora containing the agent for regulating intestinal flora constituent ratio described in any one of (48) to (51) above as an active ingredient and/or additive.

(53) A kit for improving intestinal flora, comprising the composition for improving intestinal flora described in (52) above and instructions for use.

(54) A method for producing a food or beverage, food with function claims, food for specific health use, functional nutritional food, cosmetic, quasi-drug or pharmaceutical, characterized in incorporating the agent for regulating intestinal flora constituent ratio described in any one of (47) to (51).

(55) An anti-chronic mild inflammatory agent containing processed Glycyrrhizae radix and/or processed Angelica keiskei as an active ingredient.

(56) The anti-chronic mild inflammatory agent described in (55) above, wherein the processed Glycyrrhizae radix and/or Angelica keiskei is any one of those selected from the group consisting of a solvent extract of the root, stem and sap of Glycyrrhizae radix and/or Angelica keiskei, the sap of Glycyrrhizae radix and/or Angelica keiskei, and a concentrate of the sap of Glycyrrhizae radix and/or Angelica keiskei.

(57) The anti-chronic mild inflammatory agent described in (55) or (56) above for incorporating in a food or beverage, food with function claims, food for specific health use, functional nutritional food, cosmetic, quasi-drug or pharmaceutical.

(58) The anti-chronic mild inflammatory agent described in any one of (55) to (57) above, for maintaining healthy obesity.

(59) A composition for maintaining healthy obesity containing the anti-chronic mild inflammatory agent described in any one of (55) to (58) above as an active ingredient and/or additive.

(60) A kit for maintaining healthy obesity comprising the composition for maintaining healthy obesity described in (59) above and instructions for use.

(61) A method for producing a food or beverage, food with function claims, food for specific health use, functional nutritional food, cosmetic, quasi-drug or pharmaceutical, characterized in incorporating the anti-chronic mild inflammatory agent described in any one of (55) to (58) above.

### Effects of the Invention

According to the present invention, an agent for maintaining healthy obesity can be provided that contains processed Glycyrrhizae radix and/or Angelica keiskei as an active ingredient. Since intestinal flora can be improved, chronic mild inflammation of adipose tissue can be improved, or body weight and visceral fat can be reduced by using the agent for maintaining healthy obesity of the present invention, the effects of preventing progression of obesity to lifestyle disease and maintaining healthy obesity are obtained.

According to the present invention, an agent for regulating intestinal flora constituent ratio can be provided containing processed Glycyrrhizae radix and/or Angelica keiskei as an active ingredient. Since intestinal flora is improved by using the agent for regulating intestinal flora constituent ratio of the present invention, effects such as reducing body weight, improving physical constitution, alleviating allergy symptoms, improving immune index or improving autism, and the effect of being able to prevent progression of obesity to lifestyle disease are obtained.

According to the present invention, an anti-chronic mild inflammatory agent can be provided that contains processed Glycyrrhizae radix and/or Angelica keiskei as an active ingredient. Since chronic mild inflammation of adipose tissue is improved by using the anti-chronic mild inflammatory agent of the present invention, the effect of being able to prevent progression of obesity to lifestyle disease is obtained.

According to the present invention, an agent for reducing body weight, visceral fat, subcutaneous fat or ectopic fat can be provided that contains processed Glycyrrhizae radix and/or Angelica keiskei as an active ingredient. Since body weight, visceral fat, subcutaneous fat or ectopic fat can be reduced by using the agent for reducing body weight, visceral fat, subcutaneous fat or ectopic fat of the present invention, the effect of being able to prevent progress of obesity to lifestyle disease is obtained.

### BEST MODE FOR CARRYING OUT THE INVENTION

The following provides a detailed explanation of the present invention.

Since the agent for maintaining healthy obesity according to the present invention is able to regulate the constituent ratio of intestinal flora, improve chronic mild inflammation of adipose tissue or reduce body weight and visceral fat, the progression of obesity to lifestyle disease can be prevented and healthy obesity can be maintained.

In the present invention, maintenance of healthy obesity refers to preventing exacerbation of symptoms and obesity observed in metabolic syndrome caused by obesity in non-morbidly obese persons. More specifically, this refers to preventing exacerbation of abdominal obesity (waist circumference), serum neutral fat value, serum HDL cholesterol value, blood pressure, blood sugar value and BMI value in persons having a BMI of 23 to less than 25. In addition, academically this refers to preventing exacerbation of insulin resistance in persons with high-normal BMI values.

The agent for regulating intestinal flora constituent ratio according to the present invention increases bacteria belonging to the phylum Bacteroidetes and decreases bacteria belonging to the phylum Firmicutes.

The agent for regulating intestinal flora constituent ratio according to the present invention can also be used as an intestinal flora ameliorant since it can be used to improve intestinal flora and the intestinal environment.

The agent for regulating intestinal flora constituent ratio according to the present invention can also be used as an agent for improving intestinal flora and/or an agent stabilizing intestinal flora since it can be used restore and/or stabilize healthy intestinal flora.

The agent for regulating intestinal flora constituent ratio according to the present invention can also be used as an agent for maintaining healthy obesity since it is able to prevent progression of obesity to lifestyle disease.

The anti-chronic mild inflammatory agent according to the present invention can be used as a crown-like structure formation inhibitor since it is able to reduce the number of crown-like structures.

The anti-chronic mild inflammatory agent according to the present invention can also be used as an adipose tissue anti-chronic mild inflammatory agent since it can be used to improve chronic mild inflammation of adipose tissue.

The anti-chronic mild inflammatory agent according to the present invention can also be used as an agent for maintaining healthy obesity since it is able to prevent progression of obesity to lifestyle disease.

The agent for reducing body weight, visceral fat, subcutaneous fat or ectopic fat according to the present invention is able to reduce body weight, visceral fat, subcutaneous fat or ectopic fat.

The agent for reducing body weight, visceral fat, subcutaneous fat or ectopic fat according to the present invention can also be used as an agent for maintaining healthy obesity since it is able to prevent progression of obesity to lifestyle disease.

The agent for regulating intestinal flora constituent ratio or anti-chronic mild inflammatory agent of the present invention is only required to contain processed Glycyrrhizae radix and/or Angelica keiskei as an active ingredient, and there are no particular limitations on the processed Glycyrrhizae radix and/or Angelica keiskei incorporated therein. For example, the processed Glycyrrhizae radix and/or Angelica keiskei may be contained irrespective of the degree of purification thereof. Here, processed Glycyrrhizae radix and/or Angelica keiskei refers to the entire plant of Glycyrrhizae radix and/or Angelica keiskei or a portion thereof, or that obtained by subjecting a liquid contained in Glycyrrhizae radix and/or Angelica keiskei (such as sap) to arbitrary processing treatment (such as drying, extraction, heating or purification treatment).

The agent for maintaining healthy obesity, the agent for regulating intestinal flora constituent ratio, the anti-chronic mild inflammatory agent and the agent for reducing body weight, visceral fat, subcutaneous fat or ectopic fat according to the present invention are characterized by the containing processed Glycyrrhizae radix as an active ingredient.

<Processed Glycyrrhizae radix>

In the present invention, the entire plant of Glycyrrhizae radix or a portion thereof (such as the root, stem, leaf, fruit (seed), flower bud, flower or bark) can be used for the processed Glycyrrhizae radix.

<Crushed (Powdered), Dried or Extracted Glycyrrhizae radix or Dried Powder (Extract Powder) Thereof>

In the present invention, examples of processed Glycyrrhizae radix include crushed (powdered), dried and extracted Glycyrrhizae radix and a dried powder (extract powder) thereof. Dried or extracted Glycyrrhizae radix refers to a crushed (powdered), dried or extracted root, stem, stolon, leaf, flower, fruit or other edible portion thereof or a dried powder (extract powder) thereof. One or more types of sites may also be used as a mixture. Crushed Glycyrrhizae radix, obtained by crushing the root, stem or stolon of Glycyrrhizae radix, is used preferably, and Glycyrrhizae radix extract (extract powder), obtained by extracting the root, stem or stolon of Glycyrrhizae radix with a solvent, is used more preferably.

Glycyrrhizae radix extract (extract powder) is obtained by drying an extract obtained by extracting the aforementioned edible portion with a solvent. The extraction solvent is selected from the group consisting of water, alcohols such as methanol or ethanol, and mixed solvents of water and an alcohol or ketones such as acetone. Water, alcohol or aqueous alcohol can be preferably used for the extraction solvent. Hot water, ethanol or aqueous ethanol can be more preferably used for the extraction solvent. The aforementioned aqueous alcohol is used at an alcohol concentration of, for example, 0.1% by weight to 99.9% by weight, preferably 10% by weight to 99.9% by weight, and more preferably 30% by weight to 70% by weight, or for example, 0.1% (v/v) to 99.9% (v/v), preferably 10% (v/v) to 99.9% (v/v), and more preferably 30% (v/v) to 70% (v/v). Examples of the drying method include, but are not limited to, spray drying and freeze-drying.

In the present invention, the processed Glycyrrhizae radix contains, for example, glycyrrhizinic acid, 22β-acetoxyglycyrrhizin, licorice saponin G2 (24-hydroxyglycyrrhizin), licorice saponin H2 (liquiritinic acid diglucoside), liquiritin, liquiritigenin, isoquiritin or isoliquiritigenin. The content of each component in the processed Glycyrrhizae radix can be measured using ordinary methods.

In the present invention, the processed Glycyrrhizae radix contains, for example, 0.5% by weight to 20.0% by weight, preferably 0.5% by weight to 8.0% by weight, and more preferably 1.0% by weight to 6.0% by weight of glycyrrhizinic acid based on the total weight of the processed Glycyrrhizae radix. Furthermore, an extract obtained by extracting Glycyrrhizae radix with an organic solvent not containing water, or an extract obtained by further extracting an extraction residue after water extraction of Glycyrrhizae radix with an organic solvent contains glycyrrhizinic acid at less than 0.5% by weight based on the total weight of the extract.

In the present invention, the processed Glycyrrhizae radix contains, for example, 0.1% by weight to 10.0% by weight and preferably 0.3% by weight to 10.0% by weight of liquiritin based on the total weight of the processed Glycyrrhizae radix.

In the present invention, the processed Glycyrrhizae radix contains, for example, 0.05% by weight to 5.00% by weight and preferably 0.10% by weight to 4.00% by weight of licorice saponin H2 based on the total weight of the processed Glycyrrhizae radix.

In the present invention, the processed Glycyrrhizae radix contains, for example, 0.01% by weight to 4.00% by weight and preferably 0.05% by weight to 2.50% by weight of liquiritigenin based on the total weight of the processed Glycyrrhizae radix.

In the present invention, the processed Glycyrrhizae radix contains, for example, 0.01% by weight to 1.50% by weight and preferably 0.08% by weight to 1.00% by weight of licorice saponin G2 based on the total weight of the processed Glycyrrhizae radix.

The agent for maintaining healthy obesity, the agent for regulating intestinal flora constituent ratio, the anti-chronic mild inflammatory agent and the agent for reducing body weight, visceral fat, subcutaneous fat or ectopic fat according to the present invention are characterized by containing processed Angelica keiskei, such as crushed Angelica keiskei, as an active ingredient.

Angelica keiskei (Ashitaba in Japanese) is plant of the Umbelliferae family, designated with the scientific name, “Angelica keiskei koidz”, found in tropical regions such as Hachijojima Island or the Izu Islands of Japan. Recently, Angelica keiskei has come to be cultivated not only in Japan, but also in various regions such as Indonesia or South Korea using seeds harvested on Hachijojima Island. Furthermore, there are no particular limitations on the source of the Angelica keiskei used in the present invention.

<Processed Angelica keiskei>

In the present invention, the entire plant of Angelica keiskei or a portion thereof (such as the root, stem, leaf, fruit (seed), flower bud, flower or bark) can be used for the processed Angelica keiskei.

<Crushed, Dried or Extracted Angelica keiskei or Dried Powder (Extract Powder) Thereof>

In the present invention, examples of processed Angelica keiskei include crushed, dried and extracted Angelica keiskei and a dried powder (extract powder) thereof. Crushed or extracted Angelica keiskei refers to a crushed, dried or extracted root, stem, stolon, leaf, flower, fruit or other edible portion thereof or a dried powder (extract powder) thereof. One or more types of sites may also be used as a mixture. An extract powder extracted from the root or stem is used more preferably.

The extract powder of Angelica keiskei is obtained by drying an extract obtained by extracting the aforementioned edible portion with a solvent. The extraction solvent is selected from the group consisting of water, alcohols such as methanol or ethanol, and mixed solvents of water and an alcohol or ketones such as acetone. Water, alcohol or aqueous alcohol is used preferably. Hot water, ethanol or aqueous ethanol is used more preferably for the extraction solvent. The aforementioned aqueous alcohol is used at an alcohol concentration of, for example, 0.1% by weight to 99.9% by weight, preferably 10% by weight to 99.9% by weight, and more preferably 30% by weight to 70% by weight, or for example, 0.1% (v/v) to 99.9% (v/v), preferably 10% (v/v) to 99.9% (v/v), and more preferably 30% (v/v) to 70% (v/v). Examples of the drying method include, but are not limited to, spray drying and freeze-drying.

The processed Angelica keiskei contains, for example, 7.0% by weight to 10.0% by weight, and preferably 8.0% by weight to 9.0% by weight, of Angelica keiskei chalcone or a salt thereof (such as 4-hydroxyderricin or xanthoangelol) based on the total weight of the processed Angelica keiskei. The content of each component in the processed Glycyrrhizae radix can be measured using ordinary methods.

The extract powder of Angelica keiskei preferably contains Angelica keiskei chalcone or a salt thereof (such as 4-hydroxyderricin or xanthoangelol) at a ratio of about 8% to 9%.

The sap of Angelica keiskei contains a large amount of oil, does not become a powder even if dried as it is, it aggregates even if freeze-dried, thereby making it difficult to powder. Therefore, an excipient is added to this sap. Examples of preferably used excipients include dextrin, lactose, crystalline cellulose, silicon dioxide and cyclic oligosaccharides. Among these, cyclic oligosaccharides, and particularly cyclodextrin, are superior in terms of the stability of functional components in the resulting powder and dispersibility in a solvent such as water.

The aforementioned sap and excipient are preferably mixed at a weight ratio of 7:3 to 6:4. During mixing, a suitable solvent such as ethanol can be added to ensure uniform mixing. In addition, a suitable amount of an antifoaming agent may be added to prevent bubbling. Next, the mixture is sterilized as necessary. The same method as that used to heat-sterilize the aforementioned sap can be employed for sterilization. Moreover, sterilization can also be carried out in an autoclave (such as by heating for about 20 minutes at a temperature of about 120° C.).

<Composition for Regulating or Improving Intestinal Flora Containing an Agent for Regulating Intestinal Flora Constituent Ratio as an Active Ingredient and/or an Additive>

The agent for regulating intestinal flora constituent ratio of the present invention can be used as an active ingredient and/or additive of various types of compositions for regulating or improving intestinal flora such as a food or beverage, food with function claims, food for specific health use, functional nutritional food, cosmetic, quasi-drug or pharmaceutical. Namely, by using the agent for regulating intestinal flora constituent ratio of the present invention, a composition for regulating or improving intestinal flora can be prepared that has the effect of effectively regulating or improving intestinal flora by increasing the number of bacteria belonging to the phylum Bacteroidetes and decreasing the number of bacteria belonging to the phylum Firmicutes present in intestinal flora.

<Composition for Maintaining Healthy Obesity Containing an Agent for Regulating Intestinal Flora Constituent Ratio as an Active Ingredient and/or an Additive>

The agent for regulating intestinal flora constituent ratio of the present invention can be used as an active ingredient and/or additive of various compositions for maintaining healthy obesity such as a food or beverage, food with function claims, food for specific health use, functional nutritional food, cosmetic, quasi-drug or pharmaceutical. Namely, by using the agent for regulating intestinal flora constituent ratio of the present invention, a composition for maintaining healthy obesity can be prepared that has the effect of effectively regulating or improving intestinal flora by increasing the number of bacteria belonging to the phylum Bacteroidetes and decreasing the number of bacteria belonging to the phylum Firmicutes present in intestinal flora.

<Composition for Reducing Body Weight, Visceral Fat, Subcutaneous Fat or Ectopic Fat Containing an Agent for Regulating Intestinal Flora Constituent Ratio as an Active Ingredient and/or an Additive>

The agent for regulating intestinal flora constituent ratio of the present invention can be used as an active ingredient and/or additive of various compositions for reducing body weight, visceral fat, subcutaneous fat or ectopic fat such as a food or beverage, food with function claims, food for specific health use, functional nutritional food, cosmetic, quasi-drug or pharmaceutical. Namely, by using the agent for regulating intestinal flora constituent ratio of the present invention, a composition for reducing body weight, visceral fat, subcutaneous fat or ectopic fat can be prepared that has the effect of effectively regulating or improving intestinal flora by increasing the number of bacteria belonging to the phylum Bacteroidetes and decreasing the number of bacteria belonging to the phylum Firmicutes present in intestinal flora.

<Composition for Maintaining Healthy Obesity Containing an Anti-Chronic Mild Inflammatory Agent as an Active Ingredient and/or an Additive>

The anti-chronic mild inflammatory agent of the present invention can be used as an active ingredient and/or additive of various compositions for maintaining healthy obesity such as a food or beverage, food with function claims, food for specific health use, functional nutritional food, cosmetic, quasi-drug or pharmaceutical. Namely, by using the anti-chronic mild inflammatory agent of the present invention, a composition for maintaining healthy obesity can be prepared that has the effect of effectively improving chronic mild inflammation of adipose tissue by reducing the number of crown-like structures.

<Composition for Reducing Body Weight, Visceral Fat, Subcutaneous Fat or Ectopic Fat Containing an Anti-Chronic Mild Inflammatory Agent as an Active Ingredient and/or an Additive>

The anti-chronic mild inflammatory agent of the present invention can be used as an active ingredient and/or additive of various compositions for reducing body weight, visceral fat, subcutaneous fat or ectopic fat such as a food or beverage, food with function claims, food for specific health use, functional nutritional food, cosmetic, quasi-drug or pharmaceutical. Namely, by using the anti-chronic mild inflammatory agent of the present invention, a composition for reducing body weight, visceral fat, subcutaneous fat or ectopic fat can be prepared that has the effect of effectively improving chronic mild inflammation of adipose tissue by reducing the number of crown-like structures.

There are no particular limitations on the incorporated amount of the agent for regulating intestinal flora constituent ratio in the composition for regulating or improving intestinal flora, the composition for maintaining healthy obesity or the composition for reducing body weight, visceral fat, subcutaneous fat or ectopic fat of the present invention, or the incorporated amount of the anti-chronic mild inflammatory agent in the composition for maintaining healthy obesity or the composition for reducing body weight, visceral fat, subcutaneous fat or ectopic fat, and is suitably set according to the purpose of application (such as the target disease or type of symptoms), target application site, gender and age of the subject, form of the food or beverage, food with function claims, food for specific health use, functional nutritional food, cosmetic, quasi-drug or pharmaceutical, administration method, ingestion method or number of administrations thereof, and preference.

There are no particular limitations on the amount of the agent for maintaining healthy obesity, the agent for regulating intestinal flora constituent ratio, the anti-chronic mild inflammatory agent or the agent for reducing body weight, visceral fat, subcutaneous fat or ectopic fat of the present invention incorporated in a food or beverage, food with function claims, food for specific health use, functional nutritional food, cosmetic, quasi-drug or pharmaceutical, and is incorporated such that, for example, the daily adult dosage of the agent for maintaining healthy obesity, the agent for regulating intestinal flora constituent ratio, the anti-chronic mild inflammatory agent or the agent for reducing body weight, visceral fat, subcutaneous fat or ectopic fat of the present invention is, for example, 0.01 g to 3.0 g and preferably 0.1 g to 1.0 g.

In addition, as was previously described, the processed Glycyrrhizae radix and/or Angelica keiskei can be obtained by, for example, extracting and purifying from Glycyrrhizae radix and/or Angelica keiskei, the extract per se of Glycyrrhizae radix and/or Angelica keiskei obtained during this process may be used as the agent for regulating intestinal flora constituent ratio or the anti-chronic mild inflammatory agent of the present invention, and in the case of using this extract per se of Glycyrrhizae radix and/or Angelica keiskei in the agent for maintaining healthy obesity, the agent for regulating intestinal flora constituent ratio, the anti-chronic mild inflammatory agent or the agent for reducing body weight, visceral fat, subcutaneous fat or ectopic fat of the present invention, the extract of Glycyrrhizae radix and/or Angelica keiskei is incorporated so that the daily adult dosage of the extract of Glycyrrhizae radix and/or Angelica keiskei for a food or beverage, food with function claims, food for specific health use, functional nutritional food, cosmetic, quasi-drug or pharmaceutical is, for example, 0.01 g to 3.0 g and preferably 0.1 g to 1.0 g.

In the case of preparing the composition for regulating or improving intestinal flora, the composition for maintaining healthy obesity or the composition for reducing body weight, visceral fat, subcutaneous fat or ectopic fat of the present invention as a food or beverage, the food or beverage is prepared in a desired form by mixing in a sweetener, colorant, preservative, thickener, stabilizer, gelling agent, sizing agent, antioxidant, coloring agent, bleaching agent, antifungal agent (anti-mold agent), yeast food, gum base, fragrance, sour agent, flavoring agent, emulsifier, pH adjuster, brine, swelling agent, nutritional supplement or other food or beverage material in addition to the agent for maintaining healthy obesity, agent for regulating intestinal flora constituent ratio, anti-chronic mild inflammatory agent or agent for reducing body weight, visceral fat, subcutaneous fat or ectopic fat of the present invention. In the case of using the composition for improving or regulating intestinal flora of the present invention in the form of a food or beverage, there are no particular limitations on the form thereof. Examples of the forms thereof include supplement-type foods such as gels, granules, grains, capsules, tablets, powders, liquids or semi-solids, beverages such as carbonated beverages, soft drinks, milk drinks, alcoholic beverages, fruit juice beverages, teas, nutritional drinks, powdered beverages such as powdered juice or powdered soup, confections such as chewing gum, tablets, candies, cookies, gumdrops, crackers, biscuits or jelly, as well as bread, noodles, cereal, jam and condiments. These food products can be used as foods or beverages for regulating or improving intestinal flora (for regulating the constituent ratio of intestinal flora), for maintaining healthy obesity, or for reducing body weight, visceral fat, subcutaneous fat or ectopic fat, and for example, can be used as ordinary foods or beverages as well as nutritional supplements, foods with function claims, foods for specified health uses or nutriceuticals such as foods for the sick.

In the case of preparing the composition for regulating or improving intestinal flora, the composition for maintaining healthy obesity or the composition for reducing body weight, visceral fat, subcutaneous fat or ectopic fat of the present invention as a pharmaceutical (including quasi-drugs), the pharmaceutical may arbitrarily incorporate other pharmaceutically effective ingredients or pharmaceutically allowable carriers or additives as necessary in addition to the agent for maintaining healthy obesity, the agent for regulating intestinal flora constituent ratio, the anti-chronic mild inflammatory agent, the composition for maintaining healthy obesity, or the agent for reducing body weight, visceral fat, subcutaneous fat or ectopic fat of the present invention. Specific examples of pharmaceutically acceptable carriers and additives include binders, disintegrating agents, lubricants, wetting agents, buffers, preservatives and fragrances. In the case of preparing the agent for regulating intestinal flora constituent ratio of the present invention as a pharmaceutical, there are no particular limitations on the form thereof. Examples of the forms thereof include injection preparations, external preparations, inhalants, suppositories, films, troches, liquids, powders, tablets, granules, capsules, syrups, eye drops, eye washes and nose drops. Among these, forms suitable for oral administration (namely, pharmaceuticals for internal use) are preferable, and examples of such forms include troches, liquids, powders, tablets, capsules and syrups. These pharmaceuticals (including quasi-drugs) are used as pharmaceuticals for regulating or improving intestinal flora (or regulating intestinal flora constituent ratio), maintaining healthy obesity or reducing body weight, visceral fat, subcutaneous fat or ectopic fat.

In the case of preparing the composition for regulating or improving intestinal flora, the composition for maintaining healthy obesity or the composition for reducing body weight, visceral fat, subcutaneous fat or ectopic fat of the present invention as a cosmetic (including functional cosmetics) or quasi-drug for external use, a pharmaceutically or cosmetologically allowed carrier (such as water or oily component) is incorporated therein in a desired form in addition to the agent for maintaining healthy obesity, the agent for regulating intestinal flora constituent ratio, the anti-chronic mild inflammatory agent or the agent for reducing body weight, visceral fat, subcutaneous fat or ectopic fat of the present invention. There are no particular limitations on the aforementioned cosmetic provided it can be applied to skin. Examples of forms thereof include liquids, milky liquids, powders, solids, suspensions, creams, ointments, mousses, granules, tablets, gels, jellies, pastes, gels, aerosols, sprays, liniments and packs. These cosmetics are used as cosmetics for regulating or improving intestinal flora (or for regulating the constituent ratio of intestinal flora), for maintaining healthy obesity, or for reducing body weight, visceral fat, subcutaneous fat or ectopic fat.

The composition for regulating or improving intestinal flora of the present invention can be used to restore and/or stabilize healthy intestinal flora by increasing the number of bacteria belonging to the phylum Bacteroidetes and reducing the number of bacteria belonging to the phylum Firmicutes.

The composition for maintaining healthy obesity of the present invention can be used to effectively improve chronic mild inflammation of adipose tissue and sever the link between obesity and lifestyle disease by reducing the number of crown-like structures.

The present invention also relates to a kit for improving intestinal flora that contains the composition for regulating or improving intestinal flora of the present invention and instructions for the use thereof.

The present invention also relates to a kit for maintaining healthy obesity that contains the composition for maintaining healthy obesity of the present invention and instructions for the use thereof.

The instructions for use contained in the kit for regulating or improving intestinal flora of the present invention may contain a description stating that processed Glycyrrhizae radix and/or Angelica keiskei increases bacteria belonging to the phylum Bacteroidetes and decreases bacteria belonging to the phylum Firmicutes, that processed Glycyrrhizae radix and/or Angelica keiskei is an active ingredient of the agent for regulating intestinal flora constituent ratio, and that the agent for regulating intestinal flora constituent ratio or composition for improving intestinal flora containing the agent for regulating intestinal flora constituent ratio as an active ingredient (such as a food or beverage, food with function claims, food for specific health use, functional nutritional food, cosmetic, quasi-drug or pharmaceutical) is useful in restoring and/or stabilizing healthy intestinal flora and improving an obese constitution.

The instructions for use contained in the kit for maintaining healthy obesity of the present invention may contain a description stating that processed Glycyrrhizae radix and/or Angelica keiskei reduces the number of crown-like structures, that processed Glycyrrhizae radix and/or Angelica keiskei is an active ingredient of an anti-chronic mild inflammatory agent, that a composition for maintaining healthy obesity containing an anti-chronic mild inflammatory agent as an active ingredient (such as a food or beverage, food with function claims, food for specific health use, functional nutritional food, cosmetic, quasi-drug or pharmaceutical) effectively improves chronic mild inflammation of adipose tissue, and can be used to sever the link between obesity and lifestyle disease. In addition, the instructions for use may also contain a description stating that processed Glycyrrhizae radix and/or Angelica keiskei increases bacteria belonging to the phylum Bacteroidetes and decreases bacteria belonging to the phylum Firmicutes, that processed Glycyrrhizae radix and/or Angelica keiskei is an active ingredient of the agent for regulating intestinal flora constituent ratio, and that the agent for regulating intestinal flora constituent ratio or composition for maintaining healthy obesity containing the agent for regulating intestinal flora constituent ratio as an active ingredient (such as a food or beverage, food with function claims, food for specific health use, functional nutritional food, cosmetic, quasi-drug or pharmaceutical) is useful in restoring and/or stabilizing healthy intestinal flora and improving an obese constitution.

The instructions for use contained in the kit for improving intestinal flora or the kit for maintaining healthy obesity are inserted in a package in the form of an explanatory statement such as academic article, patent specification or insert, is written on a package in the form of instructions, can be acquired in the form of an explanatory statement from a website by accessing a URL written on the package, or may be disclosed in the form of instructions on a website.

### EXAMPLES

Although the following provides a more detailed explanation of the present invention by indicating examples thereof, the scope of the present invention is not limited thereto.

### Preparation Example 1: Processed Angelica keiskei (Angelica keiskei Extract Containing about 8% to 9% of Chalcones)

When Angelica keiskei native to Hachijojima was cultivated and grown until the sprout reached a height of 50 cm to 60 cm during the period from one year after seeding to prior to blooming, the base of the sprout was cut to harvest the sap that exuded therefrom. Seventy g of this sap and 30 g of cyclodextrin powder were uniformly mixed, and the resulting mixture was freeze-dried to obtain 45 g of powder containing an Angelica keiskei component. This powder containing an Angelica keiskei component contained chalcones in the form of 4-hydroxyderricin and xanthoangelol at a ratio of 4.02% by weight and 4.68% by weight, respectively. The total contained amount of these chalcones was 8.70% by weight, thereby obtaining a powder containing an Angelica keiskei component having a high chalcone content. Hereinafter, processed Angelica keiskei in the form of the product of mixing the sap of Angelica keiskei with cyclodextrin powder followed by freeze-drying is also referred to as “Angelica keiskei polyphenol” (total chalcone content: 8% to 9%, Japan Bioscience Laboratory Co., Ltd.).

### Preparation Example 2: Processed Angelica keiskei (Angelica keiskei Extract Containing about 8% to 9% of Chalcones)

Preparation Example 2 was carried out in the same manner as Preparation Example 1 with the exception of using crystalline cellulose instead of cyclodextrin powder. As a result, a powder containing an Angelica keiskei component was obtained that was nearly the same as that of Preparation Example 1.

Furthermore, the amount of chalcone contained in the powder containing an Angelica keiskei component was measured according to the method indicated below.

One hundred mg of powder containing an Angelica keiskei component is accurately weighed in a stoppered test tube followed by the addition of 10 ml of ethyl acetate and subjecting to ultrasonic extraction for 20 minutes, adding 1 ml of water and shaking well for 1 minute. After allowing to stand briefly, a portion of the ethyl acetate layer is filtered with a Cosmonice Filter W (0.45 μm, Nacalai Tesque Inc.) and used as sample solution. Quantification is carried out by HPLC using the Cosmosil 5C18-AR (Nacalai Tesque Inc.) for the column, using a mixture of MeOH and water (4:1) for the mobile phase and injecting 10 μl for the sample volume under conditions of a column temperature of 50° C. and UV detection at a wavelength of 330 nm. The retention time of 4-hydroxyderricin is about 10 minutes and the retention time of xanthoangelol is about 11 minutes.

Preparation Example of Processed Glycyrrhizae radix

The stem, root and stolon of Glycyrrhizae radix were crushed to obtain Glycyrrhizae radix powder for use as processed Glycyrrhizae radix. Fifty g of Glycyrrhizae radix powder were extracted at 20° C. using 500 mL of ethanol containing 30% to 99.5% water, and the resulting extract was dried by freeze-drying and with an evaporator to obtain 30%, 50%, 70% and 99.5% ethanol extracts of Glycyrrhizae radix.

### <Test Example 1> Effects of Processed Angelica keiskei

Five-week-old male C57BL/6J mice were divided into groups of ten animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with processed Angelica keiskei. Body weight and visceral fat weight (total weight of periepididymal fat, perirenal fat and mesenteric fat) were measured after ingesting the feed for 8 weeks. The proportions of bacteria belonging to the phylum Firmicutes and bacteria belonging to the phylum Bacteroidetes in the intestinal flora after ingesting the feed for 8 weeks were each measured by terminal restriction fragment length polymorphism analysis (T-RFLP, Nagashima method). Furthermore, the processed Angelica keiskei was fed to the animals after mixing in 0.2% of Angelica keiskei polyphenol (total chalcone content: 8% to 9%, Japan Bioscience Laboratory Co., Ltd.). The proportions of bacteria belonging to the phyla Bacteroidetes and Firmicutes in the intestinal flora of the mice (based on a value of 100% for the total number of bacteria) are shown in Table 1.

TABLE 1
Changes in Proportions of Bacteroidetes and Firmicutes Bacteria
in Mouse Intestinal Flora, Body Weight and Visceral Fat Weight

| | | | Proportion of | | Proportion of | | Ratio of Firmicutes | |
|---|---|---|---|---|---|---|---|---|
| | | Test | Bacteroidetes | | Firmicutes | | Bacteria/Bacteroidetes | |
| | Feed | Substance | Bacteria (%) | | Bacteria (%) | | Bacteria | |
| Example 1 | HFS | Angelica | 28.7 ± 1.4 | * | 59.4 ± 1.9 | n.s. | 2.13 ± 0.15 | ** |
| | | keiskei | | | | | | |
| | | polyphenol | | | | | | |
| Comparative | MF | None | 37.3 ± 3.0 | ** | 49.5 ± 3.6 | ** | 1.52 ± 0.27 | ** |
| Example 1 | | | | | | | | |
| Comparative | HFS | None | 20.8 ± 2.1 | vs. | 65.7 ± 3.3 | vs. | 3.70 ± 0.62 | vs. |
| Example 2 | | | | | | | | |

| | | | |
|---|---|---|---|
| | Test | | Visceral Fat |
| Feed | Substance | Body Weight (g) | Weight (g) |

| | | | | | | |
|---|---|---|---|---|---|---|
| Example 1 | HFS | Angelica | 27.8 ± 0.5 | ** | 1.67 ± 0.10 | * |
| | | keiskei | | | | |
| | | polyphenol | | | | |
| Comparative | MF | None | 27.4 ± 0.8 | ** | 1.26 ± 0.16 | ** |
| Example 1 | | | | | | |
| Comparative | HFS | None | 30.9 ± 0.7 | vs. | 2.56 ± 0.19 | vs. |
| Example 2 | | | | | | |

Mean ± standard error, data tested using Ryan's multiple comparison test (* p < 0.05, ** p < 0.01 vs. Comparative Example 2)

### <Test Example 2> Effects of Glycyrrhizae radix

Five-week-old male C57BL/6J mice were divided into groups of five animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with Glycyrrhizae radix powder or Glycyrrhizae radix ethanol extract. Body weight and visceral fat weight (total weight of periepididymal fat, perirenal fat and mesenteric fat) were measured after ingesting the feed for 8 weeks. The proportions of bacteria belonging to the phylum Firmicutes and bacteria belonging to the phylum Bacteroidetes in the intestinal flora after ingesting the feed for 8 weeks were each measured by terminal restriction fragment length polymorphism analysis (T-RFLP, Nagashima method). Processed Glycyrrhizae radix was fed to the animals by mixing in 1% Glycyrrhizae radix powder and 0.1% Glycyrrhizae radix 95% ethanol extract into the feed. Changes in the proportions of bacteria belonging to the phyla Bacteroidetes and Firmicutes in the intestinal flora of the mice (based on a value of 100% for the total number of bacteria) are shown in Table 2.

TABLE 2
Changes in Proportions of Bacteroidetes and Firmicutes
Bacteria in Mouse Intestinal Flora, Body Weight and Visceral Fat Weight

| | | | Proportion of | | Proportion of | | Ratio of Firmicutes | |
|---|---|---|---|---|---|---|---|---|
| | | Test | Bacteroidetes | | Firmicutes | | Bacteria/Bacteroidetes | |
| | Feed | Substance | Bacteria (%) | | Bacteria (%) | | Bacteria | |
| Example 1 | HFS | Glycyrrhizae | 37.0 ± 2.8 | ** | 34.9 ± 5.9 | ** | 1.00 ± 0.21 | ** |
| | | radix | | | | | | |
| | | powder | | | | | | |
| Example 2 | HFS | Glycyrrhizae | 29.9 ± 1.2 | ** | 55.6 ± 2.8 | * | 1.87 ± 0.13 | ** |
| | | radix 99.5% | | | | | | |
| | | ethanol | | | | | | |
| | | extract | | | | | | |
| Comparative | MF | None | 30.9 ± 4.2 | ** | 56.1 ± 5.9 | * | 2.05 ± 0.44 | ** |
| Example 1 | | | | | | | | |
| Comparative | HFS | None | 17.9 ± 3.2 | vs. | 69.7 ± 5.6 | vs. | 4.60 ± 1.03 | vs. |
| Example 2 | | | | | | | | |

| | | | |
|---|---|---|---|
| | Test | | Visceral Fat |
| Feed | Substance | Body Weight (g) | Weight (g) |

| | | | | | | |
|---|---|---|---|---|---|---|
| Example 1 | HFS | Glycyrrhizae | 29.3 ± 0.4 | n.s. | 1.89 ± 0.11 | ** |
| | | radix | | | | |
| | | powder | | | | |
| Example 2 | HFS | Glycyrrhizae | 28.2 ± 1.3 | * | 1.87 ± 0.26 | ** |
| | | radix 99.5% | | | | |
| | | ethanol | | | | |
| | | extract | | | | |
| Comparative | MF | None | 28.4 ± 1.4 | * | 1.47 ± 0.28 | ** |
| Example 1 | | | | | | |
| Comparative | HFS | None | 31.1 ± 0.7 | vs. | 2.70 ± 0.20 | vs. |
| Example 2 | | | | | | |

Mean ± standard error, data tested using Ryan's multiple comparison test (* p < 0.05, ** p < 0.01 vs. Comparative Example 2)

### <Text Example 3> Effect of Processed Angelica keiskei on Number of Crown-Like Structures in Periepididymal Fat

Five-week-old male C57BL/6J mice were divided into groups of ten animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with Angelica keiskei polyphenol. After ingesting the feed for 8 weeks, periepididymal fat was fixed with 10% neutral buffered formalin followed by the preparation of paraffin-embedded HE-stained samples. The number of crown-like structures (per sample) in periepididymal fat after ingesting feed for 8 weeks was measured by observing tissue samples respectively stained with HE stain. Crown-like structures refer to a state in which macrophages are distributed so as to surround adipocytes. Furthermore, the processed Angelica keiskei was fed to the animals after mixing 0.2% of Angelica keiskei polyphenol (total chalcone content: 8% to 9%, Japan Bioscience Laboratory Co., Ltd.) into the feed. The numbers of crown-like structures (per sample) present in mouse periepididymal fat are shown in Table 3.

TABLE 3
Changes in Number of Crown-like Structures in
Mouse Periepididymal Fat

| | | | No. of Crown-like | |
|---|---|---|---|---|
| | | | Structures | |
| | Feed | Test Substance | (per sample) | |
| Example 1 | HFS | Angelica keiskei polyphenol | 0.90 ± 0.28 | ** |
| Comparative | MF | None | 1.80 ± 0.44 | * |
| Example 1 | | | | |
| Comparative | HFS | None | 7.10 ± 2.64 | vs. |
| Example 2 | | | | |

Mean ± standard error, data tested using Ryan's multiple comparison test (* p < 0.05, ** p < 0.01 vs. Comparative Example 2)

### <Test Example 4> Effect of Processed Glycyrrhizae radix on Number of Crown-Like Structures in Periepididymal Fat

Five-week-old male C57BL/6J mice were divided into groups of five animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with Glycyrrhizae radix powder. After ingesting the feed for 8 weeks, periepididymal fat was fixed with 10% neutral buffered formalin followed by the preparation of paraffin-embedded HE-stained samples. The number of crown-like structures (per sample) in periepididymal fat after ingesting feed for 8 weeks was measured by observing tissue samples respectively stained with HE stain. Crown-like structures refer to a state in which macrophages are distributed so as to surround adipocytes. Furthermore, the processed Glycyrrhizae radix was fed to the animals after mixing 1% of Glycyrrhizae radix powder into the feed. The numbers of crown-like structures (per sample) present in mouse periepididymal fat are shown in Table 4.

TABLE 4
Changes in Number of Crown-like Structures
in Mouse Periepididymal Fat

| | | | No. of Crown-like | |
|---|---|---|---|---|
| | | | Structures | |
| | Feed | Test Substance | (per sample) | |
| Example 1 | HFS | Glycyrrhizae radix powder | 2.00 ± 0.89 | * |
| Comparative | MF | None | 1.80 ± 0.44 | ** |
| Example 1 | | | | |
| Comparative | HFS | None | 7.10 ± 2.64 | vs. |
| Example 2 | | | | |

Feed
Test Substance
No. of Crown-like Structures (per sample)
Example 1
Glycyrrhizae radix powder
Comparative Example 1
None
Comparative Example 2
None
Mean ± standard error, data tested using Ryan's multiple comparison test (* p < 0.05, ** p < 0.01 vs. Comparative Example 2)

### <Test Example 5> Effect of Processed Angelica keiskei on Intestinal Flora

Five-week-old male C57BL/6J mice were divided into groups of five to six animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with processed Angelica keiskei. The proportions of bacteria belonging to the phylum Bacteroidetes and bacteria belonging to the phylum Firmicutes in the intestinal flora after ingesting the feed for 8 weeks were each measured by terminal restriction fragment length polymorphism analysis (T-RFLP, Nagashima method). Furthermore, the processed Angelica keiskei was fed to the animals by mixing 0.2% of Angelica keiskei polyphenol (total chalcone content: 8% to 9%, Japan Bioscience Laboratory Co., Ltd.) into the feed. The proportions of bacteria belonging to the phyla Bacteroidetes and Firmicutes in the intestinal flora of the mice (based on a value of 100% for the total number of bacteria) are shown in Table 5.

TABLE 5
Changes in Proportions of Bacteroidetes and Firmicutes
Bacteria in Mouse Intestinal Flora

| | | | | | | | Ratio of | |
|---|---|---|---|---|---|---|---|---|
| | | | Proportion of | | Proportion of | | Firmicutes | |
| | | | Bacteroidetes | | Firmicutes | | Bacteria/Bacteroidetes | |
| | Feed | Test Substance | Bacteria (%) | | Bacteria (%) | | Bacteria | |
| Example 1 | HFS | Angelica keiskei | 65.4 ± 5.2 | ** | 27.2 ± 4.5 | ** | 0.45 ± 0.09 | ** |
| | | polyphenol, 0.2% | | | | | | |
| Comparative | MF | None | 53.3 ± 4.1 | ** | 19.5 ± 3.1 | ** | 0.41 ± 0.11 | ** |
| Example 1 | | | | | | | | |
| Comparative | HFS | None | 17.9 ± 3.2 | vs. | 69.7 ± 5.6 | vs. | 4.60 ± 1.03 | vs. |
| Example 2 | | | | | | | | |

Mean ± standard error, data tested using Ryan's multiple comparison test (* p < 0.05, ** p < 0.01 vs. Comparative Example 2)

### <Test Example 6> Effect of Processed Glycyrrhizae radix on Intestinal Flora

Five-week-old male C57BL/6J mice were divided into groups of five to six animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with Glycyrrhizae radix powder or Glycyrrhizae radix ethanol extract. The proportions of bacteria belonging to the phylum Bacteroidetes and bacteria belonging to the phylum Firmicutes in the intestinal flora after ingesting the feed for 8 weeks were each measured by terminal restriction fragment length polymorphism analysis (T-RFLP, Nagashima method).

Furthermore, the processed Glycyrrhizae radix was fed to the animals after mixing 1% Glycyrrhizae radix powder into the feed, mixing 0.3% Glycyrrhizae radix 30% ethanol extract into the feed, mixing 0.3% Glycyrrhizae radix 50% ethanol extract into the feed, mixing 0.3% Glycyrrhizae radix 70% ethanol extract into the feed, or mixing 0.1% Glycyrrhizae radix 99.5%% ethanol extract into the feed. The proportions of bacteria belonging to the phyla Bacteroidetes and Firmicutes in the intestinal flora of the mice (based on a value of 100% for the total number of bacteria) are shown in Table 6.

TABLE 6
Changes in Proportions of Bacteroidetes and Firmicutes
Bacteria in Mouse Intestinal Flora

| | | | | | | | Ratio of | |
|---|---|---|---|---|---|---|---|---|
| | | | Proportion of | | Proportion of | | Firmicutes | |
| | | | Bacteroidetes | | Firmicutes | | Bacteria/Bacteroidetes | |
| | Feed | Test Substance | Bacteria (%) | | Bacteria (%) | | Bacteria | |
| Example 1 | HFS | Glycyrrhizae radix | 37.0 ± 2.8 | ** | 34.9 ± 5.9 | ** | 1.00 ± 0.21 | ** |
| | | powder, 1% | | | | | | |
| Example 2 | HFS | Glycyrrhizae radix | 68.2 ± 1.3 | ** | 16.7 ± 1.8 | ** | 0.25 ± 0.03 | ** |
| | | 30% ethanol | | | | | | |
| | | extract, 0.3% | | | | | | |
| Example 3 | HFS | Glycyrrhizae radix | 63.4 ± 2.6 | ** | 19.1 ± 3.9 | ** | 0.32 ± 0.08 | ** |
| | | 50% ethanol | | | | | | |
| | | extract, 0.3% | | | | | | |
| Example 4 | HFS | Glycyrrhizae radix | 61.6 ± 2.9 | ** | 19.7 ± 2.9 | ** | 0.32 ± 0.05 | ** |
| | | 70% ethanol | | | | | | |
| | | extract, 0.3% | | | | | | |
| Example 5 | HFS | Glycyrrhizae radix | 29.9 ± 1.2 | * | 55.6 ± 2.8 | * | 1.87 ± 0.13 | ** |
| | | 99.5% ethanol | | | | | | |
| | | extract, 0.1% | | | | | | |
| Comparative | MF | None | 53.3 ± 4.1 | ** | 19.5 ± 3.1 | ** | 0.41 ± 0.11 | ** |
| Example 1 | | | | | | | | |
| Comparative | HFS | None | 17.9 ± 3.2 | vs. | 69.7 ± 5.6 | vs. | 4.60 ± 10.3 | vs. |
| Example 2 | | | | | | | | |

Mean ± standard error, data tested using Ryan's multiple comparison test (* p < 0.05, ** p < 0.01 vs. Comparative Example 2)

### <Test Example 7> Effect of Processed Angelica keiskei and/or Glycyrrhizae radix on

Intestinal Bacteria Five-week-old male C57BL/6J mice were divided into groups of five to six animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with processed Angelica keiskei and Glycyrrhizae radix 50% ethanol extract either alone or in combination. The proportions of bacteria belonging to the phylum Bacteroidetes and bacteria belonging to the phylum Firmicutes in the intestinal flora after ingesting the feed for 8 weeks were each measured by terminal restriction fragment length polymorphism analysis (T-RFLP, Nagashima method). Furthermore, the processed Angelica keiskei was fed to the animals after mixing in 0.05% in Example 1 or 0.1% in Example 2 of Angelica keiskei polyphenol (total chalcone content: 8% to 9%, Japan Bioscience Laboratory Co., Ltd.). The processed Glycyrrhizae radix was fed to the animals by mixing 0.15% Glycyrrhizae radix 50% ethanol extract into the feed in Example 3. In Example 4, 0.05% Angelica keiskei polyphenol and 0.15% Glycyrrhizae radix 50% ethanol extract were mixed into the feed. In Example 5, 0.1% Angelica keiskei polyphenol and 0.15% Glycyrrhizae radix 50% ethanol extract were mixed into the feed. The proportions of bacteria belonging to the phyla Bacteroidetes and Firmicutes in the intestinal flora of the mice (based on a value of 100% for the total number of bacteria) are shown in Table 7.

TABLE 7
Changes in Proportions of Bacteroidetes and Firmicutes
Bacteria in Mouse Intestinal Flora

| | | | | | | | Ratio of | |
|---|---|---|---|---|---|---|---|---|
| | | | Proportion of | | Proportion of | | Firmicutes | |
| | | | Bacteroidetes | | Firmicutes | | Bacteria/Bacteroidetes | |
| | Feed | Test Substance | Bacteria (%) | | Bacteria (%) | | Bacteria | |
| Example 1 | HFS | Angelica keiskei | 64.2 ± 4.0 | ** | 23.1 ± 4.2 | ** | 0.39 ± 0.10 | ** |
| | | polyphenol, 0.05% | | | | | | |
| Example 2 | HFS | Angelica keiskei | 64.3 ± 3.7 | ** | 21.8 ± 4.1 | ** | 0.36 ± 0.09 | ** |
| | | polyphenol, 0.1% | | | | | | |
| Example 3 | HFS | Glycyrrhizae radix | 62.5 ± 2.1 | ** | 23.8 ± 3.4 | ** | 0.39 ± 0.07 | ** |
| | | 50% ethanol | | | | | | |
| | | extract, 0.15% | | | | | | |
| Example 4 | HFS | Angelica keiskei | 62.4 ± 2.2 | ** | 22.9 ± 2.5 | ** | 0.38 ± 0.05 | ** |
| | | polyphenol, 0.05% + | | | | | | |
| | | Glycyrrhizae | | | | | | |
| | | radix 50% ethanol | | | | | | |
| | | extract, 0.15% | | | | | | |
| Examples 5 | HFS | Angelica keiskei | 57.8 ± 4.6 | ** | 27.1 ± 4.1 | ** | 0.51 ± 0.10 | ** |
| | | polyphenol, 0.1% + | | | | | | |
| | | Glycyrrhizae radix | | | | | | |
| | | 50% ethanol | | | | | | |
| | | extract, 0.15% | | | | | | |
| Comparative | MF | None | 53.3 ± 4.1 | ** | 19.5 ± 3.1 | ** | 0.41 ± 0.11 | ** |
| Example 1 | | | | | | | | |
| Comparative | HFS | None | 17.9 ± 3.2 | vs. | 69.7 ± 5.6 | vs. | 4.60 ± 1.03 | vs. |
| Example 2 | | | | | | | | |

Mean ± standard error, data tested using Ryan's multiple comparison test (* p < 0.05, ** p < 0.01 vs. Comparative Example 2)

### <Test Example 8> Effect of Processed Angelica keiskei on Body Weight and Visceral Fat Weight

Five-week-old male C57BL/6J mice were divided into groups of 5 to 19 animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with processed Angelica keiskei. Body weight and visceral fat weight (total weight of periepididymal fat, perirenal fat and mesenteric fat) were measured after ingesting the feed for 8 weeks. Furthermore, the test substances were administered in the same manner as Test Example 5. Changes in mouse body weight and visceral fat weight are shown in Table 8.

TABLE 8
Changes in Mouse Body Weight and Visceral Fat Weight

| | | | | | Visceral fat weight | |
|---|---|---|---|---|---|---|
| | Feed | Test Substance | Body weight (g) | | (g) | |
| Example 1 | HFS | Angelica keiskei polyphenol | 27.66 ± 0.70 | ** | 1.71 ± 0.17 | ** |
| | | 0.2% | | | | |
| Comparative | MF | None | 26.97 ± 0.42 | ** | 1.05 ± 0.10 | ** |
| Example 1 | | | | | | |
| Comparative | HFS | None | 31.59 ± 0.42 | vs. | 2.57 ± 0.11 | vs. |
| Example 2 | | | | | | |

Mean ± standard error, data tested using Ryan's multiple comparison test (* p < 0.05, ** p < 0.01 vs. Comparative Example 2)

### <Test Example 9> Effect of Processed Glycyrrhizae radix on Body Weight and Visceral Fat Weight

Five-week-old male C57BL/6J mice were divided into groups of 5 to 19 animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with Glycyrrhizae radix powder or Glycyrrhizae radix ethanol extract. Body weight and visceral fat weight (total weight of periepididymal fat, perirenal fat and mesenteric fat) were measured after ingesting the feed for 8 weeks. Furthermore, the test substances were administered in the same manner as Test Example 6. Changes in mouse body weight and visceral fat weight are shown in Table 9.

TABLE 9
Changes in Mouse Body Weight and Visceral Fat Weight

| | | | | | Visceral fat weight | |
|---|---|---|---|---|---|---|
| | Feed | Test Substance | Body weight (g) | | (g) | |
| Example 1 | HFS | Glycyrrhizae radix powder, | 29.31 ± 0.35 | * | 1.89 ± 0.11 | * |
| | | 1% | | | | |
| Example 2 | HFS | Glycyrrhizae radix 30% | 28.94 ± 0.74 | ** | 1.72 ± 0.16 | ** |
| | | ethanol extract, 0.3% | | | | |
| Example 3 | HFS | Glycyrrhizae radix 50% | 27.98 ± 0.64 | ** | 1.58 ± 0.13 | ** |
| | | ethanol extract, 0.3% | | | | |
| Example 4 | HFS | Glycyrrhizae radix 70% | 28.37 ± 0.71 | ** | 1.59 ± 0.16 | ** |
| | | ethanol extract, 0.3% | | | | |
| Example 5 | HFS | Glycyrrhizae radix 99.5% | 28.23 ± 1.26 | ** | 1.87 ± 0.26 | ** |
| | | ethanol extract, 0.1% | | | | |
| Comparative | MF | None | 26.97 ± 0.42 | ** | 1.05 ± 0.10 | ** |
| Example 1 | | | | | | |
| Comparative | HFS | None | 31.59 ± 0.42 | vs. | 2.57 ± 0.11 | vs. |
| Example 2 | | | | | | |

Mean ± standard error, data tested using Ryan's multiple comparison test (* p < 0.05, ** p < 0.01 vs. Comparative Example 2)

### <Test Example 10> Effect of Processed Angelica keiskei and/or Glycyrrhizae radix on Body Weight and Visceral Fat Weight

Five-week-old male C57BL/6J mice were divided into groups of 5 to 19 animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with processed Angelica keiskei and Glycyrrhizae radix 50% ethanol extract either alone or in combination. Body weight and visceral fat weight (total weight of periepididymal fat, perirenal fat and mesenteric fat) were measured after ingesting the feed for 8 weeks. Furthermore, the test substances were administered in the same manner as Test Example 7. Mouse body weights and visceral fat weights are shown in Table 10.

TABLE 10
Changes in Mouse Body Weight and Visceral Fat Weight

| | | | | | Visceral fat weight | |
|---|---|---|---|---|---|---|
| | Feed | Test Substance | Body weight (g) | | (g) | |
| Example 1 | HFS | Angelica keiskei polyphenol, | 32.80 ± 0.86 | n.s. | 2.67 ± 0.19 | n.s. |
| | | 0.05% | | | | |
| Example 2 | HFS | Angelica keiskei polyphenol, | 31.70 ± 1.27 | n.s. | 2.50 ± 0.30 | n.s. |
| | | 0.1% | | | | |
| Example 3 | HFS | Glycyrrhizae radix 50% | 30.64 ± 1.06 | n.s. | 2.13 ± 0.24 | n.s. |
| | | ethanol extract, 0.15% | | | | |
| Example 4 | HFS | Angelica keiskei polyphenol, | 29.26 ± 0.64 | * | 1.69 ± 0.17 | ** |
| | | 0.05% + Glycyrrhizae radix | | | | |
| | | 50% ethanol extract, 0.15% | | | | |
| Example 5 | HFS | Angelica keiskei polyphenol, | 27.73 ± 1.02 | ** | 1.42 ± 0.30 | ** |
| | | 0.1% + Glycyrrhizae radix | | | | |
| | | 50% ethanol extract, 0.15% | | | | |
| Comparative | MF | None | 26.97 ± 0.42 | ** | 1.05 ± 0.10 | ** |
| Example 1 | | | | | | |
| Comparative | HFS | None | 31.59 ± 0.42 | vs. | 2.57 ± 0.11 | vs. |
| Example 2 | | | | | | |

Mean ± standard error, data tested using Ryan's multiple comparison test (* p < 0.05, ** p < 0.01 vs. Comparative Example 2)

### <Test Example 11> Effect of Processed Angelica keiskei on Number of Crown-Like Structures in Periepididymal Fat

Five-week-old male C57BL/6J mice were divided into groups of 16 to 22 animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with processed Angelica keiskei. After ingesting the feed for 8 weeks, periepididymal fat was fixed with 10% neutral buffered formalin followed by the preparation of paraffin-embedded HE-stained samples. The number of crown-like structures (per sample) in periepididymal fat after ingesting feed for 8 weeks was measured by observing tissue samples respectively stained with HE stain. Crown-like structures refer to a state in which macrophages are distributed so as to surround adipocytes. Furthermore, the test substances were administered in the same manner as Example 5. The numbers of crown-like structures (per sample) present in mouse periepididymal fat are shown in Table 11.

TABLE 11
Changes in Number of Crown-like Structures
in Mouse Periepididymal Fat

| | | | No. of Crown-like | |
|---|---|---|---|---|
| | | | Structures | |
| | Feed | Test Substance | (per sample) | |
| Example 1 | HFS | Angelica keiskei | 3.06 ± 1.20 | ** |
| | | polyphenol, 0.2% | | |
| Comparative | MF | None | 2.00 ± 0.48 | ** |
| Example 1 | | | | |
| Comparative | HFS | None | 13.32 ± 3.89 | vs. |
| Example 2 | | | | |

Mean ± standard error, data tested using Ryan's multiple comparison test (* p < 0.05, ** p < 0.01 vs. Comparative Example 2)

### <Test Example 12> Effect of Processed Glycyrrhizae radix on Number of Crown-Like Structures in Periepididymal Fat

Five-week-old male C57BL/6J mice were divided into groups of 5 to 22 animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with Glycyrrhizae radix powder or Glycyrrhizae radix ethanol extract. After ingesting the feed for 8 weeks, periepididymal fat was fixed with 10% neutral buffered formalin followed by the preparation of paraffin-embedded HE-stained samples. The number of crown-like structures (per sample) in periepididymal fat after ingesting feed for 8 weeks was measured by observing tissue samples respectively stained with HE stain. Crown-like structures refer to a state in which macrophages are distributed so as to surround adipocytes. Furthermore, the test substances were administered in the same manner as Example 6. The numbers of crown-like structures (per sample) present in mouse periepididymal fat are shown in Table 12.

TABLE 12
Changes in Number of Crown-like Structures
in Mouse Periepididymal Fat

| | | | No. of Crown-like | |
|---|---|---|---|---|
| | | | Structures | |
| | Feed | Test Substance | (per sample) | |
| Example 1 | HFS | Glycyrrhizae radix | 2.00 ± 0.89 | * |
| | | powder, 1% | | |
| Example 2 | HFS | Glycyrrhizae radix 30% | 6.00 ± 3.01 | n.s. |
| | | ethanol extract, 0.3% | | |
| Example 3 | HFS | Glycyrrhizae radix 50% | 4.17 ± 1.28 | * |
| | | ethanol extract, 0.3% | | |
| Example 4 | HFS | Glycyrrhizae radix 70% | 3.00 ± 1.13 | * |
| | | ethanol extract, 0.3% | | |
| Example 5 | HFS | Glycyrrhizae radix 99.5% | 5.60 ± 1.47 | n.s. |
| | | ethanol extract, 0.1% | | |
| Comparative | MF | None | 2.00 ± 0.48 | ** |
| Example 1 | | | | |
| Comparative | HFS | None | 13.32 ± 3.89 | vs. |
| Example 2 | | | | |

Mean ± standard error, data tested using Ryan's multiple comparison test (* p < 0.05, ** p < 0.01 vs. Comparative Example 2)

### <Test Example 13> Effect of Processed Angelica keiskei and/or Glycyrrhizae radix on Number of Crown-Like Structures in Periepididymal Fat

Five-week-old male C57BL/6J mice were divided into groups of 6 to 22 animals each and housed for 8 weeks while providing access to MF Diet (Oriental Yeast Co., Ltd.), high-fat, high-sugar powdered feed (abbreviated as HFS, D12079BM, Research Diets, Inc.) or HFS mixed with processed Angelica keiskei and Glycyrrhizae radix 50% ethanol extract either alone or in combination. After ingesting the feed for 8 weeks, periepididymal fat was fixed with 10% neutral buffered formalin followed by the preparation of paraffin-embedded HE-stained samples. The number of crown-like structures (per sample) in periepididymal fat after ingesting feed for 8 weeks was measured by observing tissue samples respectively stained with HE stain. Crown-like structures refer to a state in which macrophages are distributed so as to surround adipocytes. Furthermore, the test substances were administered in the same manner as Example 7. The numbers of crown-like structures (per sample) present in mouse periepididymal fat are shown in Table 13.

TABLE 13
Changes in Number of Crown-like Structures in Mouse Periepididymal Fat

| | | | No. of Crown-like | |
|---|---|---|---|---|
| | | | Structures | |
| | Feed | Test Substance | (per sample) | |
| Example 1 | HFS | Angelica keiskei polyphenol, 0.05% | 12.50 ± 3.37 | n.s. |
| Example 2 | HFS | Angelica keiskei polyphenol, 0.1% | 10.67 ± 4.11 | n.s. |
| Example 3 | HFS | Glycyrrhizae radix 50% ethanol | 6.17 ± 2.56 | n.s. |
| | | extract, 0.15% | | |
| Example 4 | HFS | Angelica keiskei polyphenol, 0.05% + | 4.33 ± 2.08 | * |
| | | Glycyrrhizae radix 50% ethanol | | |
| | | extract, 0.15% | | |
| Example 5 | HFS | Angelica keiskei polyphenol, 0.1% + | 4.33 ± 1.82 | * |
| | | Glycyrrhizae radix 50% ethanol | | |
| | | extract, 0.15% | | |
| Comparative | MF | None | 2.00 ± 0.48 | ** |
| Example 1 | | | | |
| Comparative | HFS | None | 13.32 ± 3.89 | vs. |
| Example 2 | | | | |

Mean ± standard error, data tested using Ryan's multiple comparison test (* p < 0.05, ** p < 0.01 vs. Comparative Example 2)

### INDUSTRIAL APPLICABILITY

The agent for maintaining healthy obesity of the present invention is expected to demonstrate the effect of preventing the progression of obesity to lifestyle disease and maintain healthy obesity since the use thereof improves intestinal flora, improves chronic mild inflammation of adipose tissue and reduces body weight and visceral fat.

Use of the agent for regulating intestinal flora constituent ratio of the present invention makes it possible to improve the constituent ratio of intestinal bacteria, and more specifically, increase the number of bacteria belonging to the phylum Bacteroidetes and decrease the number of bacteria belonging to the phylum Firmicutes. As a result, the agent for regulating intestinal flora constituent ratio of the present invention is expected to reduce body weight, improve physical constitution, alleviate allergy symptoms and improve immune index.

Use of the anti-chronic mild inflammatory agent of the present invention makes it possible to reduce the number of crown-like structures. As a result, the anti-chronic mild inflammatory agent of the present invention is expected to effectively improve chronic mild inflammation of adipose tissue and sever the link between obesity and lifestyle disease.

The agent for reducing body weight, visceral fat, subcutaneous fat or ectopic fat of the present invention is expected to demonstrate the effect of preventing progression of obesity to lifestyle disease since the use thereof makes it possible to reduce body weight, visceral fat, subcutaneous fat or ectopic fat.

1-35. (canceled)

36. A method for maintaining healthy obesity, comprising administering to a subject in need thereof an effective amount of at least one composition selected from the group consisting of Glycyrrhizae radix extract extracted with aqueous ethanol, Glycyrrhizae radix powder, and Angelica keiskei extract.

37. A method for improving an intestinal microflora constituent ratio, comprising administering to a subject in need thereof an effective amount of at least one composition selected from the group consisting of Glycyrrhizae radix extract extracted with aqueous ethanol, Glycyrrhizae radix powder, and Angelica keiskei extract.

38. A method for suppressing chronic mild inflammatory, comprising administering to a subject in need thereof an effective amount of at least one composition selected from the group consisting of Glycyrrhizae radix extract extracted with aqueous ethanol, Glycyrrhizae radix powder, and Angelica keiskei extract.

39. A method for reducing body weight, visceral fat, subcutaneous fat, or ectopic fat, comprising administering to a subject in need thereof an effective amount of at least one composition selected from the group consisting of Glycyrrhizae radix extract extracted with aqueous ethanol, Glycyrrhizae radix powder, and Angelica keiskei extract.

40. The method according to claim 36, wherein the Glycyrrhizae radix extract extracted with aqueous ethanol is an extract of Glycyrrhizae radix extracted with aqueous ethanol having an ethanol concentration in a range of from 30% to 70% (v/v).

41. The method according to claim 37, wherein the Glycyrrhizae radix extract extracted with aqueous ethanol is an extract of Glycyrrhizae radix extracted with aqueous ethanol having an ethanol concentration in a range of from 30% to 70% (v/v).

42. The method according to claim 38, wherein the Glycyrrhizae radix extract extracted with aqueous ethanol is an extract of Glycyrrhizae radix extracted with aqueous ethanol having an ethanol concentration in a range of from 30% to 70% (v/v).

43. The method according to claim 39, wherein the Glycyrrhizae radix extract extracted with aqueous ethanol is an extract of Glycyrrhizae radix extracted with aqueous ethanol having an ethanol concentration in a range of from 30% to 70% (v/v).

44. The method according to claim 36, wherein the Angelica keiskei extract contains Angelica keiskei chalcones at a concentration in a range of from 7.0 wt % to 10.0 wt %.

45. The method according to claim 37, wherein the Angelica keiskei extract contains Angelica keiskei chalcones at a concentration in a range of from 7.0 wt % to 10.0 wt %.

46. The method according to claim 38, wherein the Angelica keiskei extract contains Angelica keiskei chalcones at a concentration in a range of from 7.0 wt % to 10.0 wt %.

47. The method according to claim 39, wherein the Angelica keiskei extract contains Angelica keiskei chalcones at a concentration in a range of from 7.0 wt % to 10.0 wt %.
